SEQUENCE LISTING (15 sequences)

SEQ ID NO: 1 - protein, 29 aa, Artificial Sequence
  Description of Artificial Sequence Synthetic peptide
GHRPXXXXXXXXPXXXPXPPPXXXXGYRX

SEQ ID NO: 2 - protein, 29 aa, Artificial Sequence
  Description of Artificial Sequence Synthetic peptide
GHRPLDKKREEAPSLRPAPPPISGGGYRX

SEQ ID NO: 3 - protein, 25 aa, Artificial Sequence
  Description of Artificial Sequence Synthetic peptide
GHRPLDKKREEAPSLRPAPPPISGG

SEQ ID NO: 4 - protein, 25 aa, Artificial Sequence
  Description of Artificial Sequence Synthetic peptide
GHRPLDKKREEAPSLRPAPPPISGG

SEQ ID NO: 5 - protein, 26 aa, Artificial Sequence
  Description of Artificial Sequence Synthetic peptide
GHRPLDKKREEAPSLRPAPPPISGGG

SEQ ID NO: 6 - protein, 26 aa, Artificial Sequence
  Description of Artificial Sequence Synthetic peptide
GHRPLDKKREEAPSLRPAPPPISGGG

SEQ ID NO: 7 - protein, 27 aa, Artificial Sequence
  Description of Artificial Sequence Synthetic peptide
GHRPLDKKREEAPSLRPAPPPISGGGY

SEQ ID NO: 8 - protein, 27 aa, Artificial Sequence
  Description of Artificial Sequence Synthetic peptide
GHRPLDKKREEAPSLRPAPPPISGGGY

SEQ ID NO: 9 - protein, 26 aa, Artificial Sequence
  Description of Artificial Sequence Synthetic peptide
GHRPLDKKREEAPSLRPAPPPISGGC

SEQ ID NO: 10 - protein, 26 aa, Artificial Sequence
  Description of Artificial Sequence Synthetic peptide
GHRPLDKKREEAPSLRPAPPPISGGC

SEQ ID NO: 11 - protein, 27 aa, Artificial Sequence
  Description of Artificial Sequence Synthetic peptide
GHRPLDKKREEAPSLRPAPPPISGGGC

SEQ ID NO: 12 - protein, 27 aa, Artificial Sequence
  Description of Artificial Sequence Synthetic peptide
GHRPLDKKREEAPSLRPAPPPISGGGC

SEQ ID NO: 13 - protein, 28 aa, Artificial Sequence
  Description of Artificial Sequence Synthetic peptide
GHRPLDKKREEAPSLRPAPPPISGGGYC

SEQ ID NO: 14 - protein, 28 aa, Artificial Sequence
  Description of Artificial Sequence Synthetic peptide
GHRPLDKKREEAPSLRPAPPPISGGGYC

SEQ ID NO: 15 - protein, 29 aa, Artificial Sequence
  Description of Artificial Sequence Synthetic peptide
GHRPXXXXXXXXPXXXPXPPPXXXXGYRX

(19) United States
(12) Patent Application Publication
(10) Pub. No.: US 2009/0286725 A1
(43) Pub. Date: Nov. 19, 2009

(54) PEPTIDES AND DERIVATIVES THEREOF, THE MANUFACTURING THEREOF AS WELL AS THEIR USE FOR PREPARING A THERAPEUTICALLY AND/OR PREVENTIVELY ACTIVE PHARMACEUTICAL COMPOSITION

(75) Inventors: Peter PETZELBAUER, Wien (AT); Sonja REINGRUBER, Wien (AT); Waltraud PASTEINER, Pyhra (AT); Rainer HENNING, Uetliburg (CH)

Correspondence Address: Workman Nydegger;1000 Eagle Gate Tower, 60 East South Temple, Salt Lake City, UT, 84111, US

(73) Assignee: FIBREX MEDICAL RESEARCH & DEVELOPMENT GMBH, Wien (AT)

(21) Appl. No.: 12/121,544

(22) Filed: May 15, 2008

(51) Int. Cl. A61K 38/16; C07K 14/00; A61P 29/00; C07K 1/00

(52) U.S. Cl. 514/12; 530/324; 530/333

(57) ABSTRACT

Peptides and peptide derivatives of the following general Formula I:

(SEQ ID NO: 15) (I)

H₂N-GHRPX₁X₂X₃X₄X₅X₆X₇X₈PX₉X₁₀X₁₁PX₁₂PPPX₁₃X₁₄X₁₅
X₁₆GYR-X₁₇,

wherein:
X₁-X₁₆ denote one of the 20 genetically encoded amino acids,
X₁₇ denotes OR₁, with R₁=hydrogen or (C₁-C₁₀-alkyl), or
   NR₂R₃, R₂ and R₃ being identical or different and denoting hydrogen, (C₁-C₁₀)-alkyl, or a residue -PEG_{5-60K}, wherein the PEG-residue is linked to the N atom via a spacer, or
   a residue NH—Y-Z-PEG₅₋₆₀K, wherein Y denotes a chemical bond or a genetically coded amino acid from among the group of S, C, K or R, and Z denotes a spacer by way of which a polyethylene glycol (PEG)-residue may be linked,
      as well as the physiologically acceptable salts thereof,
      or wherein:
X₁₅ or X₁₆ denotes an amino acid from the group of C or K, which is linked to a residue Z-PEG_{5-60K} via the heteroatom in the side chain, and wherein
X₁₇ denotes OR₁, with R₁=hydrogen or (C₁-C₁₀-alkyl), or
   NR₂R₃, R₂ and R₃ being identical or different and denoting hydrogen or (C₁-C₁₀)-alkyl,
      as well as the physiologically acceptable salts thereof.

The present invention relates to peptides, and derivatives thereof, to the manufacturing thereof as well as to their use for preparing a therapeutically and/or preventively active drug and to such a pharmaceutical drug.

EP1586586 describes the use of peptides from the sequence of fibrin possessing anti-inflammatory effects.

Said effect may be based on the fact that the fibrin and fibrin fragments generated during the breakdown thereof bind to endothelial cells via its neo-N-terminus of the Bbeta-chain and to cells in the bloodstream via the sequence of the Aalpha-chain, thereby leading to the adhesion and transmigration of these cells into the tissue. The binding partner of the fibrin and fibrin fragments to the endothelial cells is the protein vascular endothelial (VE) cadherin, which is expressed exclusively in the adherens junction between neighboring endothelial cells. The peptides according to the invention block this interaction and thereby counteract the transmigration of blood cells. The natural defense against infections by the leukocytes in the blood is not adversely effected, however. Thus, the composition of the same, such as granulocytes, lymphocytes and monocytes, remains unaffected so that the natural defense process is maintained.

Fibrinogen is produced in the liver and, in this form, is biologically inactive and normally is provided in the blood at concentrations of around 3 g/l. Proteolytic cleavage of the proenzyme prothrombin results in the formation of thrombin, which cleaves off the fibrinopeptides A and B from the fibrinogen. In this way, fibrinogen is transformed into its biologically active form. Fibrin and fibrin cleavage products are generated.

Thrombin is formed whenever blood coagulation is activated, i.e. with damage to the tissue, be it of inflammatory, traumatic or degenerative genesis. The formation of fibrin as mediated by thrombin is basically a protective process aimed at quickly sealing any defects caused to the vascular system. However, the formation of fibrin also is a pathogenic process. The appearance of a fibrin thrombus as the triggering cause of cardiac infarction is one of the most prominent problems in human medicine.

The role which fibrin plays during the extravasation of inflammatory cells from the bloodstream into the tissue, which, on the one hand, is a desired process for the defense against pathogenic microorganisms or tumor cells in the tissue, but, on the other hand, is a process which, by itself, induces or prolongs damage done to the tissue, has so far not been examined at all or not to a sufficient extent. Fibrin binds to endothelial cells via its neo-N-terminus of Bbeta by means of the sequence to Bbeta and to cells in the bloodstream by means of the sequence Aalpha, thereby leading to the adhesion and transmigration of cells into the tissue.

By way of the mechanism described above the peptides or proteins according to the invention may prevent the adhesion of cells from the bloodstream to endothelial cells of the vascular wall and/or their subsequent transmigration from the blood into the tissue.

One of the principal abnormalities associated with acute inflammatory disease is the loss of endothelial barrier function. Structural and functional integrity of the endothelium is required for maintenance of barrier function and if either of these is compromised, solutes and excess plasma fluid leak through the monolayer, resulting in tissue oedema and migration of inflammatory cells. Many agents increase monolayer permeability by triggering endothelial cell shape changes such as contraction or retraction, leading to the formation of intercellular gaps (Lum & Malik, Am. J. Physiol. 267: L223-L241 (1994). These agents include e.g thrombin, bradykinin and vascular endothelial growth factor (VEGF).

Hyperpermeability of the blood vessel wall permits leakage of excess fluids and protein into the interstitial space. This acute inflammatory event is frequently allied with tissue ischemia and acute organ dysfunction. Thrombin formed at sites of activated endothelial cells (EC) initiates this microvessel barrier dysfunction due to the formation of large paracellular holes between adjacent EC (Carbajal et al, Am J Physiol Cell Physiol 279: C195-C204, 2000). This process features changes in EC shape due to myosin light chain phosphorylation (MLCP) that initiates the development of F-actin-dependent cytoskeletal contractile tension (Garcia et al, J Cell Physiol. 1995;163:510-522 Lum & Malik, Am J Physiol Heart Circ Physiol. 273(5): H2442-H2451. (1997).

Thrombin-induced endothelial hyperpermeability may also be mediated by changes in cell-cell adhesion (Dejana J. Clin. Invest. 98: 1949-1953 (1996). Endothelial cell-cell adhesion is determined primarily by the function of vascular endothelial (VE) cadherin (cadherin 5), a Ca-dependent cell-cell adhesion molecule that forms adherens junctions. Cadherin 5 functionis regulated from the cytoplasmic side through association with the accessory proteins b-catenin, plakoglobin (g-catenin), and p120 that are linked, in turn, to a-catenin (homologous to vinculin) and the F-actin cytoskeleton.

VE-cadherin has emerged as an adhesion molecule that plays fundamental roles in microvascular permeability and in the morphogenic and proliferative events associated with angiogenesis (Vincent et al, Am J Physiol Cell Physiol, 286(5): C987-C997 (2004). Like other cadherins, VE-cadherin mediates calcium-dependent, homophilic adhesion and functions as a plasma membrane attachment site for the cytoskeleton. However, VE-cadherin is integrated into signaling pathways and cellular systems uniquely important to the vascular endothelium. Recent advances in endothelial cell biology and physiology reveal properties of VE-cadherin that may be unique among members of the cadherin family of adhesion molecules. For these reasons, VE-cadherin represents a cadherin that is both prototypical of the cadherin family and yet unique in function and physiological relevance. A number of excellent reviews have addressed the contributions of VE-cadherin to vascular barrier function, angiogenesis, and cardiovascular physiology.

Evidence is accumulating that the VE-cadherin-mediated cell-cell adhesion is controlled by a dynamic balance between phosphorylation and dephosphorylation of the junctional proteins including cadherins and catenins. Increased tyrosine phosphorylation of b-catenin resulted in a dissociation of the catenin from cadherin and from the cytoskeleton, leading to a weak adherens junction (AJ). Similarly, tyrosine phosphorylation of VE-cadherin and b-catenin occurred in loose AJ and was notably reduced in tightly confluent monolayers (Tinsley et al., J Biol Chem, 274, 24930-24934 (1999).

In addition the correct clustering of VE-cadherin monomers in adherens junctions is indispensable for a correct signalling activity of VE-cadherin, since cell bearing a chimeric mutant (IL2-VE) containing a full-length VE-cadherin cytoplasmic tail is unable to cause a correct signalling despite its ability to bind to beta-catenin and p120 (Lampugnani et al, Mol. Biol. of the Cell, 13, 1175-1189 (2002). Rho GTPases are a family of small GTPases with profound actions on the actin cytoskeleton of cells. With respect to the functioning of the vascular system they are involved in the regulation of cell shape, cell contraction, cell motility and cell adhesion. The three most prominent family members of the Rho GTPases are RhoA, Rac and cdc42. Activation of RhoA induces the formation of f-actin stress fibres in the cell, while Rac and cdc42 affect the actin cytoskeleton by inducing membrane ruffles and microspikes, respectively (Hall, Science, 279:509-514.1998). While Rac and cdc42 can affect MLCK activity to a limited extent via activation of protein PAK (Goeckeler et al. J. Biol. Chem., 275, 24, 18366-18374 (2000), RhoA has a prominent stimulatory effect on actin-myosin interaction by its ability to stabilize the phosphorylated state of MLC (Katoh et al., Am. J. Physiol. Cell. Physiol. 280, C1669-C1679 (2001). This occurs by activation of Rho kinase that in its turn inhibits the phosphatase PP1M that hydrolyses phosphorylated MLC. In addition, Rho kinase inhibits the actin-severing action of cofilin and thus stabilizes f-actin fibres (Toshima et al., Mol. Biol. of the Cell. 12, 1131-1145 (2001). Furthermore, Rho kinase can also be involved in anchoring the actin cytoskeleton to proteins in the plasma membrane and thus may potentially act on the interaction between junctional proteins and the actin cytoskeleton (Fukata et al. Cell Biol 145:347-361 (1999).

Thrombin can activate RhoA via Gαl2/13 and a so-called guanine nucleotide exchange factor (GEF) (Seasholtz et al; Mol: Pharmacol. 55, 949-956 (1999). The GEF exchanges RhoA-bound GDP for GTP, by which RhoA becomes active. By this activation RhoA is translocated to the membrane, where it binds by its lipophilic geranyl-geranyl-anchor.

RhoA can be activated by a number of vasoactive agents, including lysophosphatidic acid, thrombin and endothelin. The membrane bound RhoA is dissociated from the membrane by the action of a guanine dissociation inhibitor (GDI) or after the action of a GTPase-activating protein (GAP). The guanine dissociation inhibitors (GDIs) are regulatory proteins that bind to the carboxyl terminus of RhoA.

GDIs inhibit the activity of RhoA by retarding the dissociation of GDP and detaching active RhoA from the plasma membrane. Thrombin directly activates RhoA in human endothelial cells and induces translocation of RhoA to the plasma membrane. Under the same conditions the related GTPase Rac was not activated. Specific inhibition of RhoA by C3 transferase from Clostridium botulinum reduced the thrombin-induced increase in endothelial MLC phosphorylation and permeability, but did not affect the transient histamine-dependent increase in permeability (van Nieuw Amerongen et al. Circ Res. 1998;83:1115-11231 (1998).

The effect of RhoA appears to be mediated via Rho kinase, because the specific Rho kinase inhibitor Y27632 similarly reduced thrombin-induced endothelial permeability.

Rac1 and RhoA have antagonistic effects on endothelial barrier function. Acute hypoxia inhibits Rac1 and activates RhoA in normal adult pulmonary artery endothelial cells (PAECs), which leads to a breakdown of barrier function (Wojciak-Stothard and Ridley, Vascul Pharmacol., 39:187-99 (2002). PAECs from piglets with chronic hypoxia induced pulmonary hypertension have a stable abnormal phenotype with a sustained reduction in Rac1 and an increase in RhoA activitity. These activities correlate with changes in the endothelial cytoskeleton, adherens junctions and permeability. Activation of Rac1 as well as inhibition of RhoA restored the abnormal phenotype and permeability to normal (Wojciak-Stothard et al., Am. J. Physiol, Lung Cell Mol. Physiol. 290, L1173-L1182 (2006).

Substances that active Rac1 and reduce RhoA activity to a level that is observed in endothelial cells in normal and stable conditions can therefore be expected to reduce endothelial hyperpermeability and have a beneficial therapeutic effect in a number of diseases. Preferably this effect is caused by a stabilization of the clustering of VE-cadherin in the adherens junction. An important component of the intracellular complex of proteins linked to VE-cadherin is fyn, a kinase which is a member of the src tyrosine kinases. The binding of the compounds which are subject to this invention to VE-cadherin cause a dissociation of fyn from VE-cadherin, which in turn leads to deactivation of thrombin induced active RhoA.

WO9216221 describes polypeptides which are covalently linked to long-chain polymers, as for instance methoxy-polyethylene glycol (PEG). The binding of polyp eptides to such polymers frequently results in a prolongation of the biological half-life of these polypeptides and delays their renal excretion. A summary of these properties may be found in Davis et al., Polymeric Materials Pharmaceuticals for Biomedical Use, pp. 441-451 (1980) The addition of PEG-groups exerts this effect in a way proportional to the molecular weight of the PEGylated peptide, as, up to a certain size of the molecule, the glomular filtration rate is inversely proportional to the molecular weight. WO2004/101600 also describes new poly(ethylene glycol)-modified compounds and their use, in particular with emphasis on modified peptides activating the erythropoietin receptor.

Further examples for the covalent modification of peptides and proteins PEG residues are interleukins (Knauf et al., J. Biol Chem. 1988, 263, 15064; Tsutumi et al., J. Controlled Release 1995, 33, 447), Interferons (Kita et al., Drug Delivery Res. 1990, 6 157), Catalase (Abuchowski et al., J. Biol. Chem. 1997, 252, 3582). A review of the prior art may be found in Reddy, Ann. of Pharmacotherapy, 2000, 34, 915.

A prolonged biological half-life is advantageous for various therapeutic uses of peptides. This is in particular true in cases of chronic diseases where the administration of the active agent over a prolonged period of time is indicated. With such indications this may improve the patient's compliance, as applying the active agent once a day will for instance be accepted more easily than continuous infusion. Apart from increasing the molecular mass by covalent modification, a prolongation of the persistency of polypeptides may be obtained by modifying them in such a way that their degradation by proteolytic enzymes (e.g. exo- or endoproteases or peptidases) is prevented.

Using various examples it has been shown that it is necessary to customize the appropriate modification for each peptide so as to prevent a significant influence on the pharmacodynamic effect as compared to the unmodified peptide. In this context the following may be referred to: Calcitonin (Lee et al. Pharm. Res. 1999, 16, 813), Growth Hormone Releasing Hormone (Esposito et al., Advanced Drug Delivery Reviews, 2003, 55, 1279), Glucagon like peptide 1 (Lee et al., Bioconjugate Res. 2005, 16, 377), as well as the growth hormone-receptor antagonist Pegvisomant (Ross et al., J. Clin. Endocrin. Metab. 2001, 86, 1716). The reviews by Caliceti and Veronese (Adv. Drug Deliv. Rev. 2003, 55 1261) and by Harris and Chess (Nature Rev. Drug Discovery 2003, 2, 214) discuss that in case of designing peptide- or protein-PEG-conjugates it is necessary to take into consideration the structure of the original substance, the molecular weight of the peptide and the polymer, the number of conjugated polymer chains as well as the linker chemistry, so as to obtain an effective peptide-PEG-conjugate.

Surprisingly it has now been found that peptides derived from the chain of the Bbeta(15-42)fibrin fragment, but which were shortened from the C-terminus by one, two or three amino acids, as well as derivatives modified at the C-terminal end of the peptide sequence also have strong anti-inflammatory and endothelium stabilizing effects. The same applies to peptides and derivatives thereof, the modification of which prevents their destruction by proteases or peptidases, as well as to peptide-PEG-conjugates and -PEG-conjugates generally derived from the basic sequence of the Bbeta(15-42)fibrin fragment, but lacking the amino acids 6 to 11

Thus the invention relates to peptides and modified peptides and which are derived from the chain of the Bbeta(15-42)-fibrin fragment, wherein one or more of the three amino acids denoted by the positions 40, 41, and 42 of the fibrin sequence have been eliminated. They may exist as free peptides or as C-terminal derivative and/or being linked to a polyethylene glycol (PEG)-polymer, and have anti-inflammatory and/or endothelium stabilizing effects. Esters or amides may for instance be taken into consideration as C-terminal derivatives.

The inventive compounds may have conservative substitutions of amino acids as compared to the natural sequence of fibrin of the warm blooded animals to be treated in one or several positions. A conservative substitution is defined as the side chain of the respective amino acid being replaced by a side chain of similar chemical structure and polarity, the side chain being derived from a genetically coded or not genetically coded amino acid. Families of amino acids of this kind having similar side chains are known in the art. They comprise for instance amino acids having basic side chains (lysins, arginins, histidine), acidic side chains (aspartic acid, glutamic acid), uncharged polar side chains (glycine, aspartamic acid, glutamine, serine, threonine, tyrosine, cysteine), non-polar side chains (alanine, valine, leucine, isoleucine, proline, phenylalanine, methionine, tryptophan), beta-branched side chains (threonine, valine, isoleucine) and aromatic side chains (tyrosine, phenylalanine, tryptophane, histidine). Such conservative substitutions of side chains may preferably be carried out in non-essential positions. In this context, an essential position in the sequence is one wherein the side chain of the relevant amino acid is of significance for its biological effect.

The invention in particular concerns peptides and peptide derivatives of the following general formula I:

(I)

H₂N-GHRPX₁X₂X₃X₄X₅X₆X₇X₈PX₉X₁₀X₁₁PX₁₂PPPX₁₃X₁₄X₁₅
X₁₆B(1)B(2)B(3)-X₁₇ ,

wherein:
B(1) denotes either a chemical bond or the amino acid G
B(2) denotes either a chemical bond or the amino acid Y
B(3) denotes either a chemical bond or the amino acid R
   X₁-X₁₆ denote one of the 20 genetically encoded amino acids,
   X₁₇ denotes OR₁ with R₁=hydrogen or (C₁-C₁₀-alkyl), or
      NR₂R₃, R₂ and R₃ being identical or different and denoting hydrogen, (C₁-C₁₀)-alkyl, or a residue -PEG_{5-60K}, wherein the PEG-residue is linked to the N atom via a spacer, or
      a residue NH—Y-Z-PEG_{5-60K}, wherein Y denotes a chemical bond or a genetically coded amino acid from among the group of S, C, K or R, and Z denotes a spacer by way of which a polyethylene glycol (PEG)-residue may be linked, as well as the physiologically acceptable salts thereof,

A preferred subject matter of the invention are peptides and peptide derivatives of the general Formula I, wherein:
B(1),B(2), and B(3) have the meaning described above
   X₁, X₉, X₁₀, X₁₄ denote L, I, S, M or A,
   X₂, X₆, X₇ denote E or D,
   X₃, X₄, X₅, X₁₁ denote R or K
   X₈, X₁₂ denote A, G, S, or L
   X₁₃ denotes I, L or V and wherein
   X₁₅, X₁₆ and X₁₇ have the same meaning as given above,
      as well as the physiologically acceptable salts thereof

A particularly preferred subject matter of the invention are peptides and peptide derivates of Formula II,

(II)
H₂N-GHRPLDKKREEAPSLRPAPPPISGG-B(1)-B(2)-B(3)-X₁₇,

wherein X₁₇ has the same meaning as given above for Formula I, as well as the physiologically acceptable salts thereof

A most highly preferred subject matter of the present invention are compounds of Formula (II), wherein
X₁₇ denotes NR₂R₃, R₂ and R₃ being identical or different and being hydrogen or (C₁-C₁₀)-alkyl, or a residue
   C(NR₂R₃)—(S-succinimido)-(PEG_{5-40K}), the succinimide residue being linked via C-atom 3 to the sulfur atom of the cysteine residue.
      as well as the physiologically acceptable salts thereof

In the above formulas I and II the following letters represent amino acid residues in accordance with the general annotation for proteins and peptides: pPhenylalanine is F, leucine is L, isoleucine is I, methionine is M, valine is V, serine is S, proline is P, threonine is T, alanine is A, tyrosine is Y, histidine is H, glutamine is Q, asparagine is N, lysine is K, aspartic acid is D, glutamic acid is E, cysteine is C, tryptophan is W, arginine is R, glycine is G.

The amino acid residues in the compounds of Formula I may either be present in their D or their L configuration.

The term peptide refers to a polymer of these amino acids, which are linked via an amide linkage.

“Physiologically acceptable” means that salts are formed with acids or bases the addition of which does not have undesirable effects when used for humans. Preferable are salts with acids or bases the use of which is listed for use with warm blooded animals, in particular humans, in the US Pharmacopoeia or any other generally recognized pharmacopoeia.

PEG stands for a polyethylene glycol residue having a molecular weight of between 5.000 and 60.000 Dalton, this molecular weight being the maximum of a molecular weight distribution, so that individual components of the mixture may have a higher or lower molecular weight.

The invention furthermore concerns processes for the production of the peptides and peptide derivatives of general Formula (I), characterized in that, either
(A) the first amino acid at the C-terminal end of the respective sequence is linked to a polymeric resin via a suitable cleavable spacer, the subsequent amino acids, optionally containing suitable protective groups for functional groups, are linked step by step according to methods known in the art, the finished peptide is cleaved off the polymeric resin according to suitable methods known in the art, the protective groups, if present, are cleaved off by suitable methods and the peptide or peptide derivative is purified according to suitable methods, or
(B) a PEG-group having a desired molecular weight is linked to a polymeric resin via a suitable spacer, the first amino acid at the N-terminal end of the peptide is linked using suitable methods, the remaining steps being the same as described in (A), or
(C) a lysine residue, containing a suitable protective group at the ε-amino group is linked to a suitable polymeric resin via a suitable spacer using suitable methods, the peptide chain is synthesized as described in (A), following cleavage from the polymeric resin and purification, if necessary, the protective group at the ε-amino group is cleaved off using suitable methods, a PEG group having a desired molecular weight is linked to the ε-amino group using a suitable activated reagent, the optionally remaining protective groups are cleaved off and the final product is purified using suitable methods, or
   (D) a peptide containing a cysteine residue is reacted with a PEG-maleimide to form compounds of Formula (III).

Suitable processing steps following (A), (B) or (C) as well as suitable reagents are for instance described in document WO 2004/101600.

Embodiments of the respective processing steps are not new per se and will be clear to an experienced specialist in the field of organic synthesis.

Processes for linking a PEG-residue to a peptide chain will be known to the skilled artisan. For instance, a cysteine (C)-residue may be reacted with PEG-maleimide, resulting in a succinimide residue as spacer for residue Z. A further possibility is reacting an optionally activated C-terminal carboxy residue with an aminoalkyl-substituted PEG residue. A further possibility is the introduction of a PEG residue by reacting an aldehyde-substituted PEG residue with the ε-amino function of a lysine residue. Activated PEG reagents having suitable spacers and reactive groups may for instance be obtained from NOF Corporation (Tokyo, Japan).

The substances according to the invention and the use of the substances according to the invention for the production of a pharmaceutical drug are of particular significance for the production of a pharmaceutical drug for the therapy of diseases resulting from the tissue-damaging effect of white blood cells, or wherein the integrity and full physiological integrity of the layer of endothelial cells lining the blood vessels is impaired.

Diseases belonging to this group are those in context with autoimmunity, as for instance collagenoses, rheumatic diseases, inflammatory bowel diseases like Morbus Crohn or Colitis ulcerosa, psoriasis and psoriatic rheumatoid arthritis, and post/parainfectious diseases as well as diseases caused by a graft-versus-host reaction. A healing effect takes place as this medical drug blocks the migration of the white blood cells into the tissue. Thus the white blood cells remain in the blood stream and cannot cause an autoreactive effect harmful to the tissue. This effect of the inventive substances is furthermore important for the treatment of shock conditions, in particular in case of septic shock triggered by infection with gram-positive or gram-negative bacterial pathogens as well as viral infections and haemorrhagic shock caused by heavy loss of blood because of severe injuries or bacterial or viral infections.

The inventive substances may generally be used in situations that can be described with the terms “Systemic Inflammatory Response Syndrome (SIRS)”, “Acute Respiratory Distress Syndrome (ARDS)” and organ- or multiorgan failure, respectively.

With a pharmaceutical drug for the therapy and/or prevention of rejection reactions of organ transplants there is a healing effect as this pharmaceutical drug prevents the migration of white blood cells from the blood stream into the donor organ, and the donor organ can therefore not be destroyed for instance by autoreactive lymphocytes.

With a pharmaceutical drug for the therapy and/or prevention of arteriosclerosis there is a healing and/or preventive effect as this pharmaceutical drug blocks the migration of lymphocytes and monocytes into the wall of the tissue and thus the activation of the cells of the tissue wall. Thus the progress of arteriosclerosis is minimized or stopped, the progredience of arteriosclerotic plaque resulting therefrom is inhibited, causing the arteriosclerosis to recede.

With a pharmaceutical drug for the therapy and/or prevention of reperfusion trauma following surgically or pharmaceutically induced re-supply with blood, e.g. following percutaneous coronary intervention, stroke, vessel surgery, cardiac bypass surgery and organ transplants, there is a healing and/or preventive effect as this pharmaceutical drug inhibits the migration of lymphocytes, neutrophils and monocytes into the wall of the vessel. Reperfusion trauma is caused by a lack of oxygen/acidosis of the cells of the vessel during its re-supply with blood, leading to their activation and/or damage. Because of this, lymphocytes, neutrophils and monocytes adhere to the vessel wall and migrate into it. Blocking the adherence and migration of lymphocytes, neutrophils and monocytes in the vessel wall causes the hypoxy/acidosis-induced damage to abate, without the subsequent inflammatory reaction causing a permanent damage to the vessel. The endothelium-stabilizing effect of the inventive compounds furthermore prevents the formation of oedemas as well as any further damage to the organs supplied via the respective blood vessels.

With a pharmaceutical drug for the therapy and/or prevention of arteriosclerosis as a consequence of metabolic diseases or the process of aging, there is a healing and/or preventive effect as this pharmaceutical drug inhibits the migration of lymphocytes, neutrophils and monocytes into the vessel wall, thus inhibiting the progredience of arteriosclerotic plaque resulting thereform.

The pharmaceutical drug according to the invention may also be used for the transportation of another drug. The inventive drug specifically binds a surface molecule on endothelial cells. Thus drugs linked thereto may be delivered to endothelial cells in high concentrations without any danger of them having side effects at other sites. An example that may be cited here is the use of substances inhibiting the division of cells, which, specifically brought to endothelial cells, may have an antiangiogenetic effect. This brings about a healing effect in tumor patients, as tumor growth is blocked by preventing the proliferation of endothelial cells and thus by preventing neoangiogenesis. The inventive compounds themselves may also develop an antiangiogenetic effect, as they, because of their endothelium-stabilizing effect, prevent the endothelial cells from changing into a proliferative phenotype and thus prevent the formation of new capillary blood vessels. Therefore they are themselves suitable for the treatment of all kinds of tumor diseases as well as the prevention and/or treatment of tumor metastases.

The inventive compounds of Formula (I) together with pharmaceutical adjuvants and additives, may be formulated into pharmaceutical preparations which also are a subject matter of the present invention. In order to prepare such formulations a therapeutically effective dose of the peptide or peptide derivative is mixed with pharmaceutically acceptable diluents, stabilizers, solubilizers, emulsifying aids, adjuvants or carriers and brought into a suitable therapeutic form. Such preparations for instance contain a dilution of various buffers (e.g. Tris-HCl, acetate, phosphate) of different pH and ionic strength, detergents and solubilizers (e.g. Tween 80, Polysorbat 80), antioxidants (e.g. ascorbic acid), and fillers (e.g. lactose, mannitol). These formulations may influence the biological availability and the metabolic behavior of the active agents.

The pharmaceutical preparations according to the invention may be administered orally, parenterally (intramuscularly, intraperitoneally, intravenously or subcutaneously), transdermally or in an erodable implant of a suitable biologically degradable polymer (e.g. polylactate or polyglycolate).

The effectiveness of the compounds according to this invention with respect to the prevention of RhoA activation and consequentially the change in the cytoskeletal structure of the endothelial cells may for instance be demonstrated by a method comprising the steps of:
a. contacting a confluent layer of cultured endothelial cells with thrombin in the presence of at least one of the test compounds
b. lysing the endothelial cells with a lysation buffer
c. measuring the RhoA activity with a specific assay, preferentially a so-called “pull down assay”.

The effectiveness in vivo may for instance be established using a model of acute pulmonitis in a rodent. The acute pulmonitis is for instance caused in mice by the intratracheal instillation of bacterial lipopolysaccharide (LPS). The effect of the active substance is measured by measuring the amount of Evans' Blue injected into the animal in pulmonory lavage or by measuring the number of extravasated leukocytes in lung lavage fluid. The inventive compounds show an effect at a dose ranging from 0.001 mg/kg body weight to 500 mg/kg body weight, preferably at a dose ranging from 0.1 mg/kg to 50 mg/kg.

A further possibility for establishing the biological effect in vivo is the reduction or complete suppression of mortality because of an infection with haemolytic viruses or bacteria. For this purpose, mice are for instance infected with a dose of Dengue viruses, wherein 50% of the animals die within a period of 5-20 days after infection. The inventive compounds bring about a reduction of this mortality at a dose ranging from 0.001 to 500 mg/kg body weight, preferably at a dose ranging from 0.1 to 50 mg/g body weight.

The following examples serve to illustrate the invention without limiting it to the examples.

General Preparation and Purification of Peptides According to the Invention

The preparation and purification of the above peptide derivatives generally takes place by way of FMOC-strategy on acid-labile resin supports using a commercially available batch peptide synthesizer as also described in the literature (e.g. “solid phase peptide synthesis—A practical approach” by E. Atherton, R.C. Sheppard, Oxford University press 1989). N-alpha-FMOC-protected derivatives, the functional side-chains of which are protected by acid-sensitive protective groups, are used as amino acid components. Unless otherwise stated, purification is carried out by means of RP-chromatography using a water/acetonitrile gradient and 0.1% TFA as ion pair reagent.

### EXAMPLE 1

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-OH

100 mg Tentagel (Rapp-Polymere) at a load of 0.24 mmol/g are transferred to a commercially available peptide synthesis device (PSMM(Shimadzu)), wherein the peptide sequence is constructed step-by-step according to the carbodiimide/HOBt method.

The FMOC-amino acid derivatives are pre-activated by adding a 5-fold equimolar excess of di-isopropy-carbodiimide (DIC), di-isopropy-ethylamine (DIPEA) und hydroxybenzotriazole (HOBt) and, following their transfer into the reaction vessel, mixed with the resin support for 30 minutes. Washing steps are carried out by 5 additions of 900 μl DMF and thorough mixing for 1 minute. Cleavage steps are carried out by the addition of 3×900 μl 30% piperidine in DMF and thorough mixing for 4 minutes.

Removal of the individual reaction and wash solutions is effected by forcing the solutions through the bottom frit of the reaction vessel.

The amino acid derivatives FMOC-Ala, FMOC-Arg(Pbf), FMOC-Asp, FMOC-Gly, FMOC-His(Trt), FMOC-Ile, FMOC-Leu, FMOC-Lys(BOC), FMOC-Pro, and FMOC-Ser(tBu) (Orpegen) are employed.

When synthesis is completed the peptide resin is dried. The peptide amide is subsequently cleaved off by treatment with trifluoracetic acid/TIS/ EDT/water (95:2:2:1 vol) for 2 hours at room temperature. By way of filtration, concentration of the solution and precipitation by the addition of ice-cold diethyl ether the crude product (75 mg) is obtained as a solid.

The peptide is purified by RP-HPLC on Kromasil RP-18 250-20, 10 μm in 0.1% TFA with a gradient of 5 on 60% acetonitrile in 40 minutes at a flow rate of 12 ml/min and evaluation of the eluate by means of a UV detector at 215 nm. The purity of the individual fractions is determined by analyt. RP-HPLC and mass spectrometry. Following combination of the purified fractions and lyophilisation 48 mg of pure product are obtained Maldi-TOF, 2458.2 m/z (m.i.).

### EXAMPLE 2

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-NH₂

100 mg Tentagel-S-RAM (Rapp-Polymere) at a load of 0.24 mmol/g are transferred to a commercially available peptide synthesis device (PSMM(Shimadzu)), wherein the peptide sequence is constructed step-by-step according to the carbodiimide/HOBt method.

The FMOC-amino acid derivatives are pre-activated by adding a 5-fold equimolar excess of di-isopropy-carbodiimide (DIC), di-isopropy-ethylamine (DIPEA) und hydroxybenzotriazole (HOBt) and, following their transfer into the reaction vessel, mixed with the resin support for 30 minutes. Washing steps are carried out by 5 additions of 900 μl DMF and thorough mixing for 1 minute. Cleavage steps are carried out by the addition of 3×900 μl 30% piperidine in DMF and thorough mixing for 4 minutes.

Removal of the individual reaction and wash solutions is effected by forcing the solutions through the bottom frit of the reaction vessel.

The amino acid derivatives FMOC-Ala, FMOC-Arg(Pbf), FMOC-Asp, FMOC-Gly, FMOC-His(Trt), FMOC-Ile, FMOC-Leu, FMOC-Lys(BOC), FMOC-Pro, and FMOC-Ser(tBu) (Orpegen) are employed.

When synthesis is completed the peptide resin is dried. The peptide amide is subsequently cleaved off by treatment with trifluoracetic acid/TIS/ EDT/water (95:2:2:1 vol) for 2 hours at room temperature. By way of filtration, concentration of the solution and precipitation by the addition of ice-cold diethyl ether the crude product (75 mg) is obtained as a solid.

The peptide is purified by RP-HPLC on Kromasil RP-18 250-20, 10 μm in 0.1% TFA with a gradient of 5 on 60% acetonitrile in 40 minutes at a flow rate of 12 ml/min and evaluation of the eluate by means of a UV detector at 215 nm. The purity of the individual fractions is determined by analyt. RP-HPLC and mass spectrometry. Following combination of the purified fractions and lyophilisation 48 mg of pure product are obtained Maldi-TOF, 2457.1 m/z (m.i.).

### EXAMPLE 3

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-OH

100 mg Tentagel (Rapp-Polymere) at a load of 0.24 mmol/g are transferred to a commercially available peptide synthesis device (PSMM(Shimadzu)), wherein the peptide sequence is constructed step-by-step according to the carbodiimide/HOBt method.

The FMOC-amino acid derivatives are pre-activated by adding a 5-fold equimolar excess of di-isopropy-carbodiimide (DIC), di-isopropy-ethylamine (DIPEA) und hydroxybenzotriazole (HOBt) and, following their transfer into the reaction vessel, mixed with the resin support for 30 minutes. Washing steps are carried out by 5 additions of 900 μl DMF and thorough mixing for 1 minute. Cleavage steps are carried out by the addition of 3×900 μl 30% piperidine in DMF and thorough mixing for 4 minutes.

Removal of the individual reaction and wash solutions is effected by forcing the solutions through the bottom frit of the reaction vessel.

The amino acid derivatives FMOC-Ala, FMOC-Arg(Pbf), FMOC-Asp, FMOC-Gly, FMOC-His(Trt), FMOC-Ile, FMOC-Leu, FMOC-Lys(BOC), FMOC-Pro, and FMOC-Ser(tBu) (Orpegen) are employed.

When synthesis is completed the peptide resin is dried. The peptide amide is subsequently cleaved off by treatment with trifluoracetic acid/TIS/ EDT/water (95:2:2:1 vol) for 2 hours at room temperature. By way of filtration, concentration of the solution and precipitation by the addition of ice-cold diethyl ether the crude product (75 mg) is obtained as a solid.

The peptide is purified by RP-HPLC on Kromasil RP-18 250-20, 10 μm in 0.1% TFA with a gradient of 5 on 60% acetonitrile in 40 minutes at a flow rate of 12 ml/min and evaluation of the eluate by means of a UV detector at 215 nm. The purity of the individual fractions is determined by analyt. RP-HPLC and mass spectrometry. Following combination of the purified fractions and lyophilisation 48 mg of pure product are obtained Maldi-TOF, 2715.1 m/z (m.i.).

### EXAMPLE 4

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-NH₂

100 mg Tentagel-S-RAM (Rapp-Polymere) at a load of 0.24 mmol/g are transferred to a commercially available peptide synthesis device (PSMM(Shimadzu)), wherein the peptide sequence is constructed step-by-step according to the carbodiimide/HOBt method.

The FMOC-amino acid derivatives are pre-activated by adding a 5-fold equimolar excess of di-isopropy-carbodiimide (DIC), di-isopropy-ethylamine (DIPEA) und hydroxybenzotriazole (HOBt) and, following their transfer into the reaction vessel, mixed with the resin support for 30 minutes. Washing steps are carried out by 5 additions of 900 μl DMF and thorough mixing for 1 minute. Cleavage steps are carried out by the addition of 3×900 μl 30% piperidine in DMF and thorough mixing for 4 minutes.

Removal of the individual reaction and wash solutions is effected by forcing the solutions through the bottom frit of the reaction vessel.

The amino acid derivatives FMOC-Ala, FMOC-Arg(Pbf), FMOC-Asp, FMOC-Gly, FMOC-His(Trt), FMOC-Ile, FMOC-Leu, FMOC-Lys(BOC), FMOC-Pro, and FMOC-Ser(tBu) (Orpegen) are employed.

When synthesis is completed the peptide resin is dried. The peptide amide is subsequently cleaved off by treatment with trifluoracetic acid/TIS/ EDT/water (95:2:2:1 vol) for 2 hours at room temperature. By way of filtration, concentration of the solution and precipitation by the addition of ice-cold diethyl ether the crude product (75 mg) is obtained as a solid.

The peptide is purified by RP-HPLC on Kromasil RP-18 250-20, 10 μm in 0.1% TFA with a gradient of 5 on 60% acetonitrile in 40 minutes at a flow rate of 12 ml/min and evaluation of the eluate by means of a UV detector at 215 nm. The purity of the individual fractions is determined by analyt. RP-HPLC and mass spectrometry. Following combination of the purified fractions and lyophilisation 48 mg of pure product are obtained Maldi-TOF, 2714.2 m/z (m.i.).

### EXAMPLE 5

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-Tyr-OH

100 mg Tentagel (Rapp-Polymere) at a load of 0.24 mmol/g are transferred to a commercially available peptide synthesis device (PSMM(Shimadzu)), wherein the peptide sequence is constructed step-by-step according to the carbodiimide/HOBt method.

The FMOC-amino acid derivatives are pre-activated by adding a 5-fold equimolar excess of di-isopropy-carbodiimide (DIC), di-isopropy-ethylamine (DIPEA) und hydroxybenzotriazole (HOBt) and, following their transfer into the reaction vessel, mixed with the resin support for 30 minutes. Washing steps are carried out by 5 additions of 900 μl DMF and thorough mixing for 1 minute. Cleavage steps are carried out by the addition of 3×900 μl 30% piperidine in DMF and thorough mixing for 4 minutes.

Removal of the individual reaction and wash solutions is effected by forcing the solutions through the bottom frit of the reaction vessel.

The amino acid derivatives FMOC-Ala, FMOC-Arg(Pbf), FMOC-Asp, FMOC-Gly, FMOC-His(Trt), FMOC-Ile, FMOC-Leu, FMOC-Lys(BOC), FMOC-Pro, FMOC-Ser(tBu) and FMOC-Tyr(tBu) (Orpegen) are employed.

When synthesis is completed the peptide resin is dried. The peptide amide is subsequently cleaved off by treatment with trifluoracetic acid/TIS/ EDT/water (95:2:2:1 vol) for 2 hours at room temperature. By way of filtration, concentration of the solution and precipitation by the addition of ice-cold diethyl ether the crude product (75 mg) is obtained as a solid.

The peptide is purified by RP-HPLC on Kromasil RP-18 250-20, 10 μm in 0.1% TFA with a gradient of 5 on 60% acetonitrile in 40 minutes at a flow rate of 12 ml/min and evaluation of the eluate by means of a UV detector at 215 nm. The purity of the individual fractions is determined by analyt. RP-HPLC and mass spectrometry. Following combination of the purified fractions and lyophilisation 48 mg of pure product are obtained Maldi-TOF, 2878.4 m/z (m.i.).

### EXAMPLE 6

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-Tyr-NH₂

100 mg Tentagel-S-RAM (Rapp-Polymere) at a load of 0.24 mmol/g are transferred to a commercially available peptide synthesis device (PSMM(Shimadzu)), wherein the peptide sequence is constructed step-by-step according to the carbodiimide/HOBt method.

The FMOC-amino acid derivatives are pre-activated by adding a 5-fold equimolar excess of di-isopropy-carbodiimide (DIC), di-isopropy-ethylamine (DIPEA) und hydroxybenzotriazole (HOBt) and, following their transfer into the reaction vessel, mixed with the resin support for 30 minutes. Washing steps are carried out by 5 additions of 900 μl DMF and thorough mixing for 1 minute. Cleavage steps are carried out by the addition of 3×900 μl 30% piperidine in DMF and thorough mixing for 4 minutes.

Removal of the individual reaction and wash solutions is effected by forcing the solutions through the bottom frit of the reaction vessel.

The amino acid derivatives FMOC-Ala, FMOC-Arg(Pbf), FMOC-Asp, FMOC-Gly, FMOC-His(Trt), FMOC-Ile, FMOC-Leu, FMOC-Lys(BOC), FMOC-Pro, and FMOC-Ser(tBu) are employed.

When synthesis is completed the peptide resin is dried. The peptide amide is subsequently cleaved off by treatment with trifluoracetic acid/TIS/ EDT/water (95:2:2: 1 vol) for 2 hours at room temperature. By way of filtration, concentration of the solution and precipitation by the addition of ice-cold diethyl ether the crude product (75 mg) is obtained as a solid.

The peptide is purified by RP-HPLC on Kromasil RP-18 250-20, 10 μm in 0.1% TFA with a gradient of 5 on 60% acetonitrile in 40 minutes at a flow rate of 12 ml/min and evaluation of the eluate by means of a UV detector at 215 nm. The purity of the individual fractions is determined by analyt. RP-HPLC and mass spectrometry. Following combination of the purified fractions and lyophilisation 48 mg of pure product are obtained Maldi-TOF, 2877.5 m/z (m.i.).

### EXAMPLE 7

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Cys-(S-succinimide-PEG_{20K})-OH

The monomeric peptide is synthesized as in Example 1, Tentagel (Rapp Polymere) being used as resin support here with FMOC-Cys(Trt) as the first amino acid.

After cleavage and purification of the peptide reaction is carried out with a 2- to 8-fold molar excess of maleinimido-PEG_{20K}. Following recovery purification is carried out on Kromasil RP-18, and the identity of the product is confirmed by way of analytical RP-HPLC and MALDI-MS.

### EXAMPLE 8

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Cys-(S-succinimido-PEG_{20K})-amide

100 mg Tentagel-S-RAM (Rapp-Polymere) at a load of 0.24 mmol/g are transferred to a commercially available peptide synthesis device (PSMM(Shimadzu)), wherein the peptide sequence is constructed step-by-step according to the carbodiimide/HOBt method.

The FMOC-amino acid derivatives are pre-activated by adding a 5-fold equimolar excess of di-isopropy-carbodiimide (DIC), di-isopropy-ethylamine (DIPEA) und hydroxybenzotriazole (HOBt) and, following their transfer into the reaction vessel, mixed with the resin support for 30 minutes. Washing steps are carried out by 5 additions of 900 μl DMF and thorough mixing for 1 minute. Cleavage steps are carried out by the addition of 3×900 μl 30% piperidine in DMF and thorough mixing for 4 minutes.

Removal of the individual reaction and wash solutions is effected by forcing the solutions through the bottom frit of the reaction vessel.

The amino acid derivatives FMOC-Ala, FMOC-Arg(Pbf), FMOC-Asp, FMOC-Gly, FMOC-His(Trt), FMOC-Ile, FMOC-Leu, FMOC-Lys(BOC), FMOC-Pro, FMOC-Ser(tBu), and FMOC-Cys(Trt) (Orpegen) are employed.

After cleavage and purification of the peptide reaction is carried out with a 2- to 8-fold molar excess of maleinimido-PEG_{20K}. Following recovery purification is carried out on Kromasil RP-18, and the identity of the product is confirmed by way of analytical RP-HPLC and MALDI-MS.

### EXAMPLE 9

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-Cys-(S-succinimide-PEG_{20K})-OH

The monomeric peptide is synthesized as in Example 1, Tentagel (Rapp Polymere) being used as resin support here with FMOC-Cys(Trt) as the first amino acid. After cleavage and purification of the peptide reaction is carried out with a 2- to 8-fold molar excess of maleinimido-PEG_{20K}. Following recovery purification is carried out on Kromasil RP-18, and the identity of the product is confirmed by way of analytical RP-HPLC and MALDI-MS.

### EXAMPLE 10

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-Cys-(S-succinimido-PEG₂₀K)-amide

100 mg Tentagel-S-RAM (Rapp-Polymere) at a load of 0.24 mmol/g are transferred to a commercially available peptide synthesis device (PSMM(Shimadzu)), wherein the peptide sequence is constructed step-by-step according to the carbodiimide/HOBt method.

The FMOC-amino acid derivatives are pre-activated by adding a 5-fold equimolar excess of di-isopropy-carbodiimide (DIC), di-isopropy-ethylamine (DIPEA) und hydroxybenzotriazole (HOBt) and, following their transfer into the reaction vessel, mixed with the resin support for 30 minutes. Washing steps are carried out by 5 additions of 900 μl DMF and thorough mixing for 1 minute. Cleavage steps are carried out by the addition of 3×900 μl 30% piperidine in DMF and thorough mixing for 4 minutes.

Removal of the individual reaction and wash solutions is effected by forcing the solutions through the bottom frit of the reaction vessel.

The amino acid derivatives FMOC-Ala, FMOC-Arg(Pbf), FMOC-Asp, FMOC-Gly, FMOC-His(Trt), FMOC-Ile, FMOC-Leu, FMOC-Lys(BOC), FMOC-Pro, FMOC-Ser(tBu), and FMOC-Cys(Trt) (Orpegen) are employed.

After cleavage and purification of the peptide reaction is carried out with a 2- to 8-fold molar excess of maleinimido-PEG_{20K}. Following recovery purification is carried out on Kromasil RP-18, and the identity of the product is confirmed by way of analytical RP-HPLC and MALDI-MS.

### EXAMPLE 11

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-Tyr-Cys-(S-succinimide-PEG_{20K})-OH

The monomeric peptide is synthesized as in Example 1, Tentagel (Rapp Polymere) being used as resin support here with FMOC-Cys(Trt) as the first amino acid.

After cleavage and purification of the peptide reaction is carried out with a 2- to 8-fold molar excess of maleinimido-PEG_{20K}. Following recovery purification is carried out on Kromasil RP-18, and the identity of the product is confirmed by way of analytical RP-HPLC and MALDI-MS.

### EXAMPLE 12

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-Tyr-Cys-(S-succinimido-PEG_{20K})-amide

100 mg Tentagel-S-RAM (Rapp-Polymere) at a load of 0.24 mmol/g are transferred to a commercially available peptide synthesis device (PSMM(Shimadzu)), wherein the peptide sequence is constructed step-by-step according to the carbodiimide/HOBt method.

The FMOC-amino acid derivatives are pre-activated by adding a 5-fold equimolar excess of di-isopropy-carbodiimide (DIC), di-isopropy-ethylamine (DIPEA) und hydroxybenzotriazole (HOBt) and, following their transfer into the reaction vessel, mixed with the resin support for 30 minutes. Washing steps are carried out by 5 additions of 900 μl DMF and thorough mixing for 1 minute. Cleavage steps are carried out by the addition of 3×900 μl 30% piperidine in DMF and thorough mixing for 4 minutes.

Removal of the individual reaction and wash solutions is effected by forcing the solutions through the bottom frit of the reaction vessel.

The amino acid derivatives FMOC-Ala, FMOC-Arg(Pbf), FMOC-Asp, FMOC-Gly, FMOC-His(Trt), FMOC-Ile, FMOC-Leu, FMOC-Lys(BOC), FMOC-Pro, FMOC-Ser(tBu), FMOC-Cys(Trt) and FMOC-Tyr(tBu) (Orpegen) are employed.

After cleavage and purification of the peptide reaction is carried out with a 2- to 8-fold molar excess of maleinimido-PEG_{20K}. Following recovery purification is carried out on Kromasil RP-18, and the identity of the product is confirmed by way of analytical RP-HPLC and MALDI-MS.

### EXAMPLE 13

The biological effect of the compounds was established in a model thrombin induced RhoA activation in human umbilical vein endothelial cell (HUVEC) culture.

HUVEC are grown to confluence under standard conditions. Before induction of Rho activity HUVEC were starved for 4 h by using IMDM (Gibco) without growth factor and serum supplements. After the starvation period 5 U/ml Thrombin (Calbiochem) or 5 U thrombin plus 50 μg/ml of test compound are added to the starvation medium for 1, 5 and 10 min. Active RhoA was isolated using Rho Assay Reagent from Upstate according to manufactures instructions. Isolates were separated on a 15% polyacrylamid gel and blotted on Nitrocellulose-Membrane (Bio-Rad). RhoA was dedected by using Anti-Rho (-A, -B, -C), clone55 from Upstate (1:500).

Relative RhoA stimulation compared to unstimulated control

| | |
|---|---|
| Control peptide 1 min | 1 |
| Control peptide 5 min | 1 |
| Control peptide 10 min | 1 |
| thrombin 5 min | 3.8 |
| thrombin + compound example 1 (10 min) | 0.85 |

1. Peptides and peptide derivatives of the following general formula I:
(SEQ ID NO: 1)

| | |
|---|---|
| (I) | H₂N-GHRPX₁X₂X₃X₄X₅X₆X₇X₈PX₉X₁₀X₁₁PX₁₂PPPX₁₃X₁₄ |
| | X₁₅X₁₆B(1)B(2)B(3)-X₁₇, |

wherein:
B(1) denotes either a chemical bond or the amino acid G
B(2) denotes either a chemical bond or the amino acid Y
B(3) denotes either a chemical bond or the amino acid R
X₁-X₁₆ denote one of the 20 genetically encoded amino acids,
X₁₇ denotes OR₁ with R₁ =hydrogen or (C₁-C₁₀-alkyl), or
NR₂R₃, R₂ and R₃ being identical or different and denoting hydrogen,
(C₁-C₁₀)-alkyl or a residue -PEG_{5-60K}, wherein the PEG-residue is linked to the N atom via a spacer, or
a residue NH-Y-Z-PEG_{5-60K}, wherein Y denotes a chemical bond or a genetically coded amino acid from among the group of S, C, K or R, and
Z denotes a spacer by way of which a polyethylene glycol (PEG)-residue may be linked, as well as the physiologically acceptable salts thereof.

2. Peptides and peptide derivatives of the general Formula I, wherein:
B(1),B(2), and B(3) have the meaning described in claim 1
X₁, X₉, X₁₀, X₁₄ denote L, I, S, M or A,
X₂, X₆, X₇ denote E or D,
X₃, X₄, X₅, X₁₁ denote R or K
X₈, X₁₂ denote A, G, S, or L
X₁₃ denotes I, L or V and wherein
X₁₅, X₁₆ and X₁₇ have the same meaning as given above,
as well as the physiologically acceptable salts thereof.

3. Peptides and peptide derivates of Formula II,
(SEQ ID NO: 2)

| | |
|---|---|
| (II) | H₂N-GHRPLDKKREEAPSLRPAPPPISGG-B(1)-B(2)-B(3)- |
| | X₁₇, |

wherein X₁₇ has the same meaning as given above for Formula I, as well as the physiologically acceptable salts thereof.
A most highly preferred subject matter of the present invention are compounds of Formula (II), wherein
X₁₇ denotes NR₂R₃, R₂ and R₃ being identical or different and being hydrogen or (C₁-C₁₀)-alkyl or a residue
C(NR₂R₃)—(S-succinimido)-(PEG_{5-40K}), the succinimide residue being linked via C-atom 3 to the sulfur atom of the cysteine residue.
as well as the physiologically acceptable salts thereof.

4. A method for manufacturing of a compound of the general formula (I), characterized in that, either
(A) the first amino acid at the C-terminal end of the respective sequence is linked to a polymeric resin via a suitable cleavable spacer, the subsequent amino acids, optionally containing suitable protective groups for functional groups, are linked step by step according to methods known in the art, the finished peptide is cleaved off the polymeric resin according to suitable methods known in the art, the protective groups, if present, are cleaved off by suitable methods and the peptide or peptide derivative is purified according to suitable methods, or
(B) a PEG-group having a desired molecular weight is linked to a polymeric resin via a suitable spacer, the first amino acid at the N-terminal end of the peptide is linked using suitable methods, the remaining steps being the same as described in (A), or
(C) a lysine residue, containing a suitable protective group at the ε-amino group is linked to a suitable polymeric resin via a suitable spacer using suitable methods, the peptide chain is synthesized as described in (A), following cleavage from the polymeric resin and purification, if necessary, the protective group at the ε-amino group is cleaved off using suitable methods, a PEG group having a desired molecular weight is linked to the ε-amino group using a suitable activated reagent, the optionally remaining protective groups are cleaved off and the final product is purified using suitable methods, or
(D) a peptide containing a cysteine residue is reacted with a PEG-maleimide to form compounds of Formula (III).

5. A pharmaceutical composition containing a compound of the general formula (I).

6. Medical use of a compound of the general formula (I).
